(19) United States
(12) Patent Application Publication
(10) Pub. No.: US 2007/0142822 A1
(43) Pub. Date: Jun. 21, 2007

(54) Method and Apparatus for Computer Controlled Metering of Liquid Medicaments in the Event of a Time Shift

(76) Inventor: Axel Remde, Lutzelfluh-Goldbach (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/613,643

(22) Filed: Dec. 20, 2006

(30) Foreign Application Priority Data
Dec. 21, 2005 (EP) 05 028 021.3

(51) Int. Cl. G06F 19/00; A61K 9/22

(52) U.S. Cl. 604/890.1; 600/300

(57) ABSTRACT

A computer-aided insulin pump by which a basal delivery profile can be automatically adapted in steps in the event of a travel-dependent time shift, in that the local time at the destination is communicated to the pump controller which uses the time, in accordance with a stored algorithm, to calculate one or more changeover delivery profiles which are then temporarily used for automatic control of the insulin pump until the changeover of time has been completed.

### CROSS-REFERENCE TO RELATED APPLICATION(S)

This application claims priority to European Patent Application No. 05028021.3, filed on Dec. 21, 2005, the content of which is incorporated in its entirety by reference herein.

### BACKGROUND

The present invention relates to devices for administering, injecting, delivering, infusing or dispensing substances, and to methods of making and using such devices. More particularly, the present invention relates to metering, control and setting apparatus and features for such devices, and methods of making, using and operating such apparatus and features. More particularly, the present invention relates to a method for operation of a metering apparatus for liquid medicaments with computer/microprocessor-aided control in the event of a time shift or displacement, e.g., a travel-dependent displacement in the local time. It further relates to the use of the method for operation of a computer-controlled delivery device, e.g., an insulin pump, to a metering apparatus for carrying out the method, and to the use of the metering apparatus for metered delivery of medicinal substances, e.g., insulin.

Automated supply of liquid medicaments to the body of a patient is often used in the case of a permanent and, over the course of a day, varying requirement for a medicament which can be administered subcutaneously. By way of example, computer-controlled insulin pumps are used for the treatment of diabetes mellitus. Such pumps allow and/or provide an insulin supply to the patient, corresponding, in good approximation, to the physiological characteristics of a non-diabetic. The so-called basal basic supply of the body with insulin is thus provided virtually continuously by the frequent, periodic delivery of very small amounts of insulin into the patient's blood circulation. Thereby the insulin delivery does not take place at a constant delivery rate but at, over the course of the day, varying delivery rates according to a diurnal profile of delivery, which is individually defined for the patient by the doctor and is stored in the pump control with reference to a pump-internal clock which is part of and/or administers control functions.

When crossing time zone boundaries because of travelling, it is preferred and/or necessary for the diurnal profile of delivery, which may also be referred to as the basal delivery profile, to be adapted to the time shift between the place of departure and the destination of the journey since, in general, daily physiological rhythm changes with changes in local time and the insulin demand profile of the traveller changes with this. To take account of the shift, the pump wearer can now, for example, set the pump-internal clock, for example at the start or end of the journey, to the local time at the destination. This setting is generally possible without any problems for minor time shifts of up to two hours. However, in the case of a journey in which a major time shift is generated within a short travelling time, for example, in the case of a flight over a plurality of time zone boundaries, this procedure is not advantageous since the physiological rhythm of the body does not adapt itself abruptly to such a time shift, but only slowly, possibly over several days. Thus, in the event of an abrupt changeover, there would initially be a considerable discrepancy between the physiological insulin demand profile, initially still largely oriented with the daily rhythm at the home location, and the real insulin delivery profile of the insulin pump.

Most doctors therefore recommend that the changeover in the diurnal profile of delivery of the insulin pump be carried out in a number of steps and over a lengthy period for major time shifts. By way of example, this is done such that the basal diurnal profile of delivery on arrival at the destination, or alternatively at the start of the journey, is shifted by two hours with respect to the “home time” and a further shift by two hours is then carried out every second day until complete adaptation to the local time at the destination is achieved. Basically, there are two different procedures for this:

a) At the appropriate times, the pump wearer advances or delays the pump-internal clock by two hours in each case. If the time shift is not an integer multiple of two hours, a shift by the residual amount is carried out in the last step.

b) On arrival, the pump clock is immediately set to the new local time. The step-by-step shift is carried out in such a way that the overall diurnal profile of delivery is in each case reprogrammed at the chosen changeover times. Even though these two methods allow the desired step-by-step change in the delivery profile, there are still considerable disadvantages.

The step-by-step changeover in the pump clock is admittedly relatively simple, but means that the displayed time does not correspond to the local time at the destination during the changeover phase. This is problematic because the pump clock is used like a wristwatch by many pump wearers and this is then not possible during the changeover phase, and can lead to irregularities in daily routine. Furthermore, it should be considered that the so-called “pump history,” i.e., the pump-internal recording of all relevant processes, such as, for example, the issue of bolus insulin at meal times, is carried out with reference to the pump clock, thus making it more difficult to evaluate the history for therapy recording purposes during the changeover phase. It is thus possible for meal-time boli to appear in the middle of the night in the pump history, for example.

Another problem is that frequent reprogramming of the basal delivery profile corresponding to the second conventional method is elaborate, susceptible to error, and not very practicable. Furthermore, with both variants there is a risk that changeovers are forgotten.

### SUMMARY

An object of the present invention is to provide methods and apparatus which do not have the disadvantages of the prior art, or at least partially avoid them.

Accordingly, a first aspect of the present invention comprises a method for operating of a computer-controlled metering apparatus for liquid medicaments, for example an insulin pump, in the event of a shift in the local time, such as a travel-dependent shift.

In one embodiment, the metering apparatus is first controlled in a first location, normally the place of departure or the home location, at which a first local time, the home time, applies, by a computer-aided control such that it delivers the liquid medicament automatically in accordance with an internally stored basal basic demand profile which is repeated daily. This demand profile may be referred to or thought of as the first diurnal profile of delivery. Starting from said first location, the metering apparatus is moved to a second location, e.g., the destination of a journey, at which a second local time applies, which differs from the first local time by a time shift, which may be referred to or thought of as the first time shift. In this case, the metering apparatus is, subsequent to the delivery of liquid medicament according to the first diurnal profile of delivery, for adaptation to the first time shift temporarily automatically controlled by the control such that it delivers the liquid medicament in accordance with one or more further diurnal profiles of delivery, which differ from the first diurnal profile of delivery and from the first diurnal profile of delivery shifted by the first time shift, in other words, in accordance with an delivery profile which does not correspond to the diurnal profile of delivery before the initiation of the changeover phase to the second local time or to the diurnal profile of delivery after completed changeover of the operation to the second local time. Thereby, the further diurnal profile or diurnal profiles of delivery is or are determined by the control in dependence on the first time shift.

In a preferred embodiment of the method according to the present invention, after the second location, the metering apparatus is moved to a further location, which may be referred to or thought of as the third location, with a third local time, which differs from the second local time by the second time shift, as is the case, for example, when the journey is continued to a further location with a different local time, or when the return journey to the place of departure, i.e., the first location, is commenced. In this case, the metering apparatus is temporarily controlled by the control for adaptation to the second time shift such that it automatically delivers the liquid medicament in accordance with one or more further diurnal profiles of delivery which differs or differ from the diurnal profile of delivery last used at the second location and from the first diurnal profile of delivery, which is shifted by the sum, with the correct mathematical sign, of the first and second time shifts. These further diurnal profiles of delivery are determined by the control or a processor in accordance with an algorithm in dependence on the second time shift. In the case of a return journey to the first location, the second time shift corresponds to the first time shift with opposite mathematical sign.

Thereby, for the case that the further diurnal profile of delivery last used at the second location differs from a diurnal profile of delivery which corresponds to the first diurnal profile of delivery shifted by the first time shift, i.e., the changeover to the second local time has not yet been entirely completed at the time of the continuation of the journey, it is advantageous for this difference to be taken into account in the determination of the further diurnal profiles of delivery which are determined in dependence on the second time shift, so that only those adaptation steps which are effectively required for adaptation of the actual delivery situation to the third local time are carried out.

Another aspect of the present invention relates to a metering apparatus for liquid medicaments, which is suitable for carrying out the method according to the present invention. The metering apparatus comprises a feed device for feeding or moving the liquid medicament from a medicament reservoir to a delivery line, for example an infusion cannula, and a computer-aided control or controller for automatic control of the feed device such that it feeds the liquid medicament automatically in accordance with a basal basic demand profile, which may be referred to as the first diurnal profile of delivery, which is stored in the control and repeated daily. The control is furthermore designed such that, upon a first time shift, one or more different further diurnal profiles of delivery, which differ from the first diurnal profile of delivery and from the first diurnal profile of delivery shifted by the first time shift, can be determined in dependence on the first time shift according to a control-internal algorithm and for the purpose of adaptation to the first shift can temporarily be used for automatic control of the feed device. By way of example, the first time shift may result from a flight from a first location with a first local time to a second location with a different, second local time, or may also be the result of a time shift from winter to summer time or be caused by a change in the diurnal rhythm of a patient, for example, when changing from daytime work to night-time work.

In a preferred embodiment of the metering apparatus according to the present invention, the control is designed such that in case of a second time shift, which follows the first time shift, at least one further diurnal profile of delivery can be determined by it according to a control-internal algorithm in dependence on the second time shift and can be used temporarily for automatic control of the metering apparatus. The at least one further diurnal profile of delivery differs from the directly previously used diurnal profile of delivery and from the first diurnal profile of delivery shifted by the sum, with the correct mathematical sign, of the first and the second time shift. The second time shift may, for example, be caused by travelling onwards to a third location with a different local time than the local time at the second location or by travelling back to the first location, i.e., reversing the first time shift.

In this case, it may be preferred for the control, or controller or processor, of the metering apparatus to be designed, additionally, such that, in the case that the diurnal profile of delivery which has last been used for adaptation to the first time shift differs from a first diurnal profile of delivery shifted by the first time shift, as is typically the case when a multistage adaptation to the first time shift has not yet been completed, this difference can optionally or automatically be taken into account by it in the determination of the further diurnal profiles of delivery which are determined in dependence on the second time shift.

The metering apparatus and operating method according to the present invention for the first time make it possible to dispense with the manual step-by-step changeover that is required in the prior art for major time shifts, thus making the operation of metering apparatuses for liquid medicaments, e.g., insulin pumps, simpler, more convenient and safer for journeys over several time zone boundaries, and, furthermore, also allowing finer changeover steps which, during the changeover phase, allow the diurnal profile of delivery to be better adapted to the physiological demand profile of the patient.

In a preferred embodiment of the method and apparatus according to the present invention, part or all of the further diurnal profiles of delivery correspond to a first diurnal profile of delivery which has been shifted in time by a specific time shift. This results in the advantage that the changeover actions by the control remain manageable and traceable, thus making it easier to check that the metering apparatus is operating correctly.

Thereby, in the case that a possible adaptation to the first time shift, which has not yet been entirely completed, is taken into account when determining further diurnal profiles of delivery in dependence on a second time shift which follows the first time shift, it is advantageous for this to be done such that the difference between the first time shift and the portion of the first time shift which has already been implemented in the last diurnal profile of delivery used for adaptation to it is added with the correct mathematical sign to the second time shift, i.e., with this being increased or decreased by this difference, and then for the further diurnal profiles of delivery to be determined in dependence on this corrected second time shift. This also makes it possible to easily and manageable link adaptation processes to time shifts that have not yet been completed without this resulting in intense discrepancies of the further diurnal profiles of delivery, which are temporarily used for control purposes, from the respective physiological demand profile.

Furthermore, in the case that the further diurnal profiles of delivery correspond to a first diurnal profile of delivery which has been shifted in time by a specific time shift, it is advantageous if the time shift difference between respectively directly successive diurnal profiles of delivery, that is to say between the first diurnal profile of delivery and the first further diurnal profile of delivery and/or between two directly successive further diurnal profiles of delivery, corresponds to an amount of time which has been obtained by integer division of the first, second or corrected second time shift used for determination of at least one of these diurnal profiles of delivery, wherein in some preferred embodiments, this amount of time is less than or equal to 4 hours, and in some preferred embodiments is less than or equal to 3 hours.

If, in this case, the time shift differences between all the directly successive diurnal profiles of delivery, which correspond to the first diurnal profile of delivery or to a first diurnal profile of delivery which has been shifted in time by a specific time shift, are identical, which may be preferred in some embodiments, then the adaptation, even to large time shifts, can be carried out in uniform steps, without involving excessive sudden correction changes.

In a preferred embodiment of the method according to the present invention, in which part or all of the further diurnal profiles of delivery correspond to a first diurnal profile of delivery, which has been shifted in time by a specific time shift, a desired or maximum permissible time shift difference between directly successive diurnal profiles of delivery which correspond to the first diurnal profile of delivery or to a first diurnal profile of delivery which has been shifted in time by a specific time shift, is communicated to the control by a manual input or via a suitable interface, which may be wireless, or is already stored in the control. The control then determines the further diurnal profiles of delivery in dependence on the first, on the second or on the corrected second time shift and on the desired or permissible time shift difference.

In an embodiment of the metering apparatus according to the present invention, in which the control is designed such that further diurnal profiles of delivery, which correspond to a first diurnal profile of delivery which has been shifted in time by a specific time shift, can be determined and can be used for automatic control by it, it is also preferred if the control is furthermore designed such that a desired or maximum permissible time shift difference between directly successive diurnal profiles of delivery which correspond to the first diurnal profile of delivery or to a first diurnal profile of delivery which has been shifted in time by a specific time shift can be communicated to the control by a manual input or via an interface, e.g., a wireless interface, or is stored in the control, and the further diurnal profiles of delivery can be determined by the control in dependence on the first or the second time shift and on the desired or permissible time shift difference. This makes it possible to influence the algorithm for determination of the further diurnal profiles of delivery in a simple manner, including with respect to the adaptation speed to be applied.

In a further preferred embodiment of the present invention, at least part of the determined further diurnal profiles of delivery correspond to a first diurnal profile of delivery which has been compressed and/or stretched in time essentially continuously variably or at one or more locations. This makes it possible to determine diurnal profiles of delivery and to use them to control the metering apparatus, which are optimally adapted to the respective physiological demand profile of the patient.

In this case, it may be preferred for these diurnal profiles of delivery to have stretches and/or compressions by a specific amount of time in each case at intervals, e.g., being stretched or compressed by a specific amount of time each hour, every 2 hours, every 4 hours or every 6 hours. In this case, depending on the application, it may be preferred for the amount of time to be identical for all the stretches and/or compressions, or else for the stretches and/or compressions to have amounts of time which differ when seen over the diurnal profile of delivery.

In some embodiments preferred embodiments, all of the determined stretched and/or compressed diurnal profiles of delivery may have an identical profile shape and to be shifted in time with respect to one another.

The last-mentioned measures make it possible to ensure that a good adaptation to the respective physiological demand profile is possible and, at the same time, that the step-by-step adaptation to the diurnal profiles of delivery remain manageable, which is advantageous from the point of view of the capability to easily check the correct operation of the metering apparatus.

In a preferred embodiment of the method according to the present invention, at least one of a desired or maximum permissible amount of time per stretch and/or compression, a desired or maximum and/or minimum permissible number of stretching and/or compression steps, and/or a desired or maximum and/or minimum permissible interval between the stretching and/or compression steps is communicated to the control by a manual input or via an interface, which may be wireless, or is already stored in the control. The further diurnal profiles of delivery are then determined by the control automatically in dependence on at least one of the first time shift and on the desired or permissible amount of time per stretch and/or compression, on the desired or maximum and/or minimum permissible number of stretching and/or compression steps, and/or on the desired or maximum and/or minimum permissible interval between the stretching and/or compression steps.

In a preferred embodiment of the metering apparatus, the control is designed such that a desired or maximum permissible amount of time per stretch and/or compression, a desired or maximum and/or minimum permissible number of stretching and/or compression steps, and/or a desired or maximum and/or minimum permissible interval between the stretching and/or compression steps can be communicated to the control by a manual input or via an interface or is stored in the control. Furthermore, the control is designed such that the further diurnal profiles of delivery can be determined by it automatically in dependence on the first or second time shift and on the desired or permissible amount of time per stretch and/or compression, on the desired or maximum and/or minimum permissible number of stretching and/or compression steps, and/or on the desired or maximum and/or minimum permissible interval between the stretching and/or compression steps.

In yet another preferred embodiment of the invention, at least part of the further diurnal profiles of delivery, which have been determined by the control, correspond to a first diurnal profile of delivery which has been shifted in time by a specific time shift and at the same time has been compressed or stretched in time. This makes it possible to adapt the method of operation of the metering apparatus optimally to the respective circumstances.

In yet another preferred embodiment of the present invention, a diurnal profile of delivery which follows a further diurnal profile of delivery which has been determined by the control corresponds to the first diurnal profile of delivery which has been shifted by the first time shift or by the sum of the first and the second time shift, which means that the adaptation to the first or to the first and to the second time shift is entirely completed.

The respective time shifts which the control takes into account to determine the further diurnal profiles of delivery are advantageously communicated to the metering apparatus or to its control, respectively, by a manual input, for example via a keyboard, by data transmission via a wire-based communication system, or by a wireless interface, for example via a radio link, by a separate program or programmer. In some preferred embodiments, the time shifts are determined by the control by evaluation of data which are received in a wireless manner, e.g., through a satellite navigation system, for example a GPS system, which is integrated in the metering apparatus.

In some preferred embodiments, the computer-aided control of the metering apparatus has at least two separate, jointly clocked clocks, one of which is used to administer the first time shift or the sum of the time shifts or a clock time which represents these time shifts, in the case of travel-dependent time shifts the second or the third local time, with this being displayed via a display on the metering apparatus. The second of the clocks is used to control the medicament delivery of the metering apparatus in accordance with a diurnal profile of delivery. In this case, the second of the clocks is set automatically by the controller in the event of a time shift to control the metering apparatus in accordance with a further diurnal profile of delivery which has been determined by the controller such that it has a time offset with respect to a base time before the first time shift, in the case of travel-dependent time shifts this is the first local time, which is less than or equal to the first time shift or to the sum of the first and the second time shift.

In this case, in embodiments of the invention in which the further diurnal profiles of delivery which have been determined by the control correspond to a first diurnal profile of delivery which has been shifted in time, it is advantageous for the second clock to be set automatically by the control such that it in each case has a time offset with respect to the base time or to the first local time, respectively, which corresponds to the time shift of the further diurnal profile of delivery which is currently being used to control the metering apparatus with respect to the first diurnal profile of delivery.

In this way, it is possible to provide metering apparatus according to the present invention which are reliable and are of simple design and which can be operated in a safe and manageable manner according to the method of the present invention.

In yet another preferred embodiment of the present invention, the degree of adaptation of the operation of the metering apparatus may be displayed graphically. More particularly, the degree of adaption may be calculated and displayed as a function of the first or second time shift or, in the case of a travel-dependent time shift, the degree of changeover of operation of the metering apparatus to a second or third local time. In other words, the degree of deviation of the further diurnal profile of delivery which is currently being used for control from the diurnal profile of delivery which, after complete adaptation or changeover, is intended for further operation of the metering apparatus, which normally corresponds to the first diurnal profile of delivery shifted by the first time shift or by the sum of the first and the second time shift, is displayed graphically at the metering apparatus. This is advantageously done in such a manner that the distances between displayed graphical elements and/or the extent of such graphical elements are used to display time differences.

In preferred embodiments, it is preferred that for the ratio of the base time, of a time which has been shifted with respect to the base time by the first time shift or the sum of the first and the second time shift, as well as the current degree of adaptation of the operation of the metering apparatus to the first or second time shift or, in the terminology used above for the case of a travel-dependent time shift, the ratio of the first local time, the second local time and/or the third local time as well as the current degree of changeover of the operation of the metering apparatus to the second or the third local time, to be displayed graphically. The display may be provided in the form of pictograms, whose distances relative to each other serve to display the respective time differences and, in particular, are proportional to the respective time differences.

It is also preferable for the current degree of adaptation of the operation of the metering apparatus to the first or second time shift or, in the case of travel-dependent time shifts, the changeover in operation of the metering apparatus to the second or third local time, to be displayed in such a manner that the current time of the second clock is displayed and/or is displayed as proportional to a time which has been shifted by the first time shift or the sum of the first and the second time shift with respect to the base time or, in the case of travel-dependent time shifts, is displayed as proportional to the second or third local time, and in some preferred embodiments, additionally to the base time or the first local time. This makes it possible to see the current operating situation of the metering apparatus at a glance at any time, thus making it easier to check its correct operation.

In yet another preferred embodiment of the present invention, the time which has been shifted by the first time shift or by the sum of the first and the second time shift with respect to the base time, or, in the case of travel-dependent time shifts, the second and/or the third local time, and additionally the base time or the first local time, can be displayed at the metering apparatus as an analogue or digital clock display.

The metering apparatus has suitable display means for the already mentioned measures for displaying times and time relationships, such as an LCD display with appropriate graphics software, which are operatively connected in a suitable manner to the control.

In yet another preferred embodiment of the metering apparatus according to the present invention it is possible to additionally use the metering apparatus to calculate boli, in the case of insulin therapy for example correction boli or meal-time boli, and to automatically take into account in the calculation corrections in dependence on the degree of adaptation of the operation of the metering apparatus to the first or second time shift or, in the case of travel-dependent time shifts, on the current degree of changeover in operation of the metering apparatus to the second or third local time.

In some embodiments, it is preferred that the corrections can be carried out or are carried out such that a major deviation from the otherwise normal target value or target value corridor of a parameter is permitted temporarily in the changeover phase. In the case of insulin therapy, by way of example, a major deviation in the blood glucose values is temporarily permitted.

In yet another preferred embodiment, control data of the metering apparatus can be stored as control history with reference to the base time or, in the case of travel-dependent time shifts, with reference to the first local time. This in contrast to the certainly likewise possible storage of the history with reference to the time which has been shifted by the first time shift or the sum of the first and the second time shift with respect to the base time, or to the second or third local time, respectively, in that the base time or the first local time, respectively, is the only time which is not affected at all by the time shift. In this case, at least in the case of the storage of the data of therapy-relevant information, for example bolus deliveries, it is additionally possible to store the time which has been shifted by the first time shift or by the sum of the first and the second time shift with respect to the base time or, in the case of travel-dependent time shifts, the second or the third local time, respectively.

Another aspect of the present invention relates to the use of the method of the present invention for operation of a computer-controlled insulin pump, in particular for supplying insulin to the body of a patient.

Another aspect of the present invention relates to the use of the metering apparatus for metered delivery of insulin, in particular to the body of a patient.

### BRIEF DESCRIPTION OF THE DRAWINGS

FIG. 1 depicts a typical and/or representative basal diurnal profile of delivery of a computer-controlled insulin pump;

FIGS. 2a-2c depict the diurnal profile of delivery of FIG. 1, in relationship to two diurnal profiles of delivery which have been shifted by different time shifts;

FIGS. 3a-3c depict the diurnal profile of delivery of FIG. 1 at continuously variable adaptation to a time shift;

FIG. 4 schematically depicts the architecture of the control or controller for a metering apparatus according to the present invention;

FIGS. 5a-5c depict the indications on a display associated with a metering apparatus according to the present invention during different states of adaptation to a time shift; and

FIG. 6 depicts a display indication as in FIG. 5a, at an opposite time shift.

### DETAILED DESCRIPTION

With regard to fastening, mounting, attaching or connecting components of the present invention, unless specifically described as otherwise, conventional mechanical fasteners and methods may be used. Other appropriate fastening or attachment methods include adhesives, welding and soldering, the latter particularly with regard to any electrical features of the present invention. Suitable electrical components and circuitry, wires, chips, boards, controllers, microprocessors, software, firmware, inputs, outputs, displays, control components, etc. may be incorporated. Generally, unless otherwise indicated, the materials for making the device of the present invention and/or its components may be selected from appropriate materials such as metals, metallic alloys, ceramics, plastics, etc.

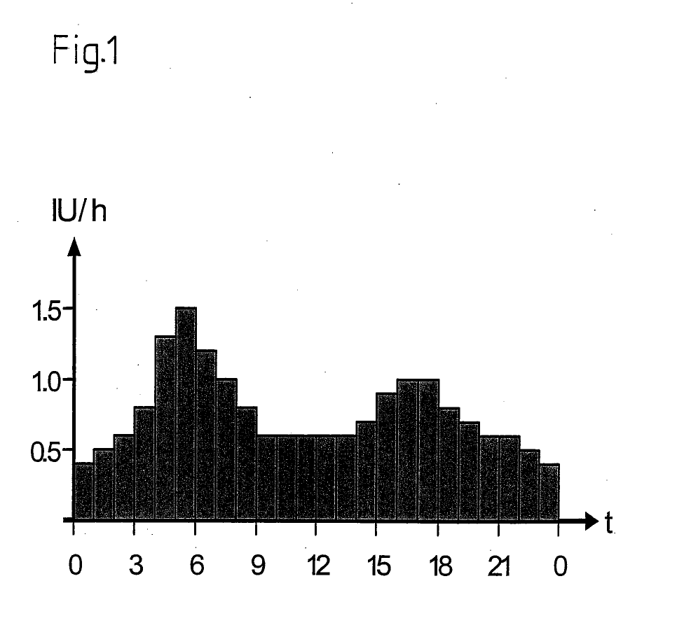

A typical diurnal profile of delivery of a computer-controlled insulin pump which can be worn at the body, also referred to as a basal delivery profile of the insulin pump, is illustrated in FIG. 1. The pump and the basal delivery serve for providing the basic supply of insulin to a diabetic. In this case, the delivery rate is plotted in insulin units per hour IU/h on the ordinate, and the time t is plotted in the scope of one day of the week with 24 hours on the abscissa. As can be seen, a continuous insulin supply is approximated by the frequent subcutaneous delivery of very small amounts of insulin to the body of the diabetic, which corresponds to the physiological insulin demand of the body. In this case, the typical delivery profile has a first maximum in the early hours of the morning, the so-called “dawn phenomenon” and a second maximum in the late afternoon, while there is a demand minimum around midnight.

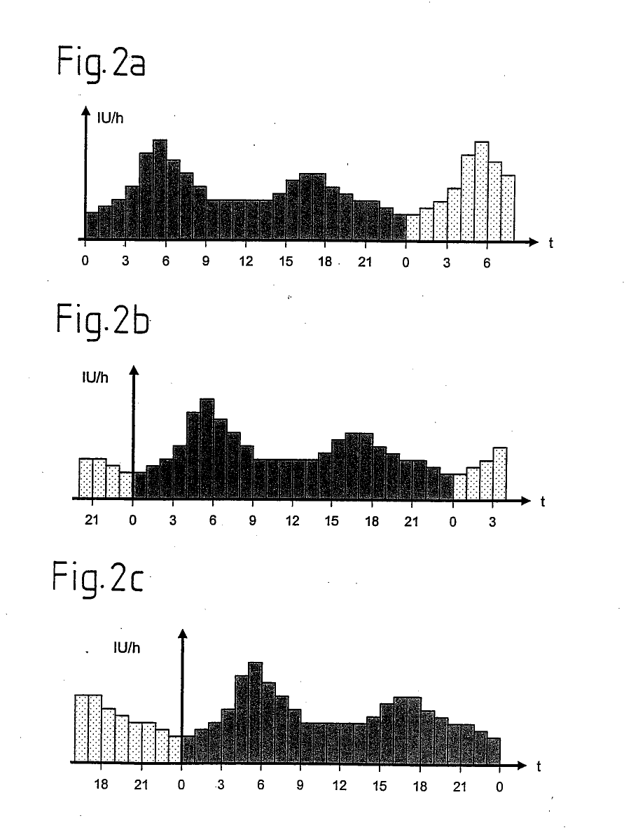

The FIGS. 2a-2c show the diurnal profile of delivery of FIG. 1, once for the local time at the home location (FIG. 2b), once time-shifted by a time shift of plus 4 hours (FIG. 2a), such as that which can occur when travelling in an eastward direction, and once time-shifted by a time shift of minus 4 hours (FIG. 2c), such as that which can occur when travelling in a westward direction, in relationship to each other. As can be seen, already a time shift of a few hours with respect to the diurnal profile of delivery used at the home location leads to a massive changeover in the individual delivery rates, for which reason most doctors recommend that adaptation to time shifts of more than 2 hours be carried out in steps.

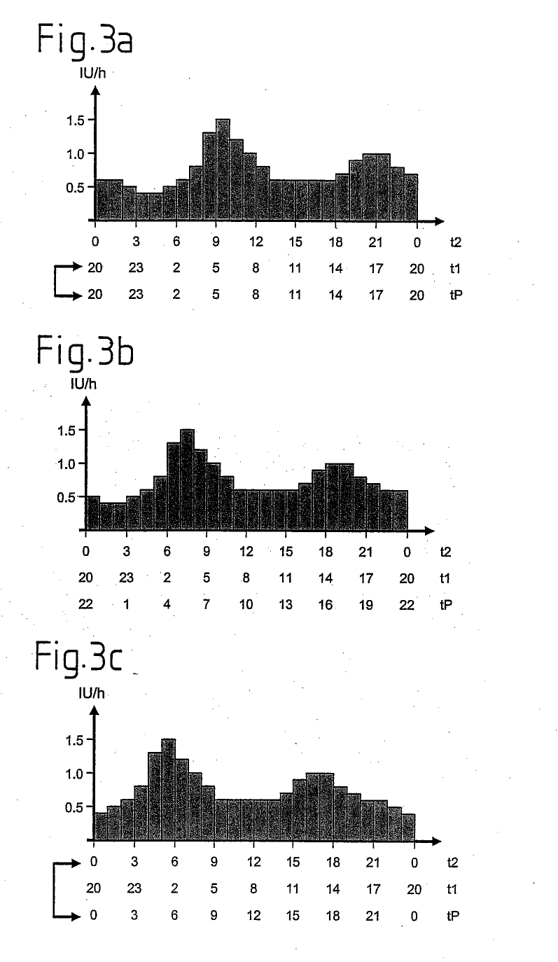

Such a stepwise changeover of the diurnal profile of delivery of a computer-controlled insulin pump from the local time t1 at the home location to a local time t2 at the destination of a journey, which has been advanced by four hours with respect to the home time t1, i.e., a stepwise changeover from the diurnal profile of delivery at the home location shown in FIG. 2b to the diurnal profile of delivery at the destination shown in FIG. 2a, which corresponds to a diurnal profile of delivery at the home location shifted by a time shift of 4 hours, is illustrated in FIGS. 3a to 3c. As can be seen, the adaptation of the delivery profile of the insulin pump in this case takes place by a stepwise changeover of the pump time tP, which is administered by the pump clock, which before the adaptation is identical to the home time t1 (see the two linked arrows in FIG. 3a), by 2 hours daily (intermediate step see FIG. 3b), until the pump time tP is identical to the local time t2 at the destination (see the two linked arrows in FIG. 3c). This stepwise adaptation (shift) in the basal diurnal profile of delivery is, at the exemplary insulin pump that is used here, automatically determined by the control in dependence on the time shift between the home location and the destination and is used for the control of the insulin delivery. Here, the changeover activity is carried out by the insulin-pump wearer at a single time, for example before the journey, during the journey or at the time of arrival at the destination. In this case, the user either declares the new local time t2 of the destination or else the time shift between the home time t1 and the destination time t2. Furthermore, the user indicates to the pump control the time steps or the adaptation rate with which the adaptation of the basal profile should be carried out, in the present case by two hours per day.

For the implementation of the time shift in the case of the exemplary insulin pump, the individual basal rates are not shifted but the basal discharge is controlled internally within the pump by means of a separate clock, which administers the pump time tP, which has a variable time offset Δt with respect to the local time t2 of the destination, also indicated on a display at the insulin pump. In some embodiments, the variable time offset corresponds at all times to the shift between the local time t2 and the diurnal profile of delivery which is currently being used to control the insulin discharge.

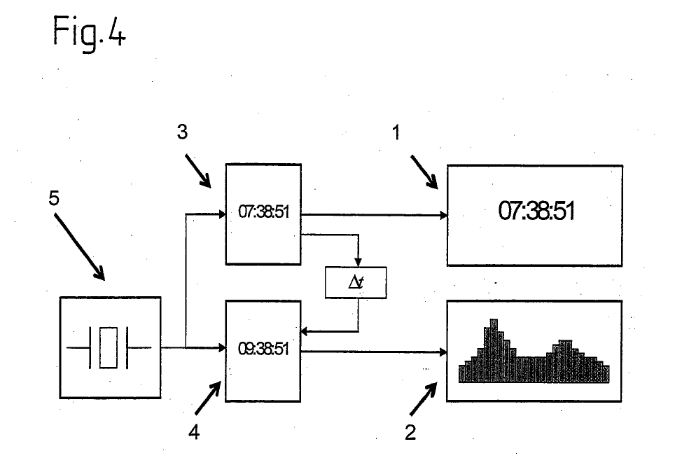

As can be seen from FIG. 4, which schematically shows the architecture of the control for an exemplary delivery device, e.g., an insulin pump, the clock time display 1 and the feed device 2 of the insulin pump are controlled via separate clocks 3, 4, which have a common clock generator 5. The first clock 3 is the central main clock of the pump control which, as has already been mentioned, administers the new local time t2 which is also displayed on the pump display 1. It is set to the new local time t2 for example at the start or end of the journey. The second clock 4 serves to control the insulin delivery via the pump time tP, which is administered by it, and which is stepwise changed over by the control to the new local time t2 (first clock 3). In principle, the overall control of the pump can take place selectively via one of the two clocks 3, 4. However, in the present case, this is done using the first clock 3, which is advantageous since this is affected by the fewest changes (only one change in each case for the outward journey and the return journey). For this reason also, the entries in the pump history are made with reference to the first clock 3. To ensure correct operation and for fault detection, respectively, the respective actual time shift Δt of the time tP of the second clock 4 with respect to the time t2 of the first clock 3 is checked at defined times, for example at every basal insulin discharge, hourly or at each change of Δt, which in the present case is done automatically by means of the pump control. Additional features, structures, functions and components of exemplary delivery devices, e.g., insulin pumps, are disclosed in U.S. Pat. No. 6,878,132, the content of which is incorporated herein by reference.

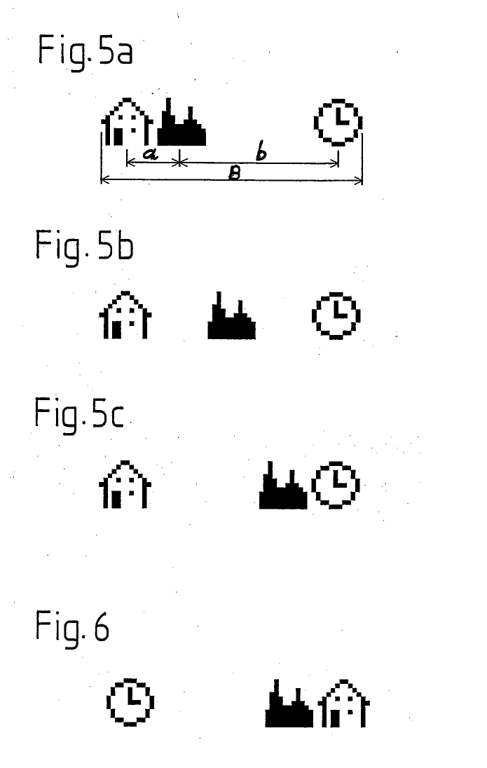

As can be seen from FIGS. 5a-5c, which show the indications on the display of an exemplary insulin pump according to the present invention during a multiple stage adaptation of the diurnal profile of delivery to a travel-dependent time shift of several hours, it is possible to display the local times at the home location and at the destination. It is also possible to display the time shift which forms the basis for the diurnal profile of delivery that is currently being used relative to the local times in relation to each other such that these are displayed by means of pictograms (“home” for the local time at the home location; “discharge profile” for the actual time shift of the diurnal profile of delivery that is currently being used; “clock” for the local time at the destination). “Home” and “clock” are in this case arranged such that they make use of virtually the display width. In the case of the positive time shift being present the pictogram “clock” is shown on the right and the pictogram “home” on the left in the display. The pictogram “discharge profile” is shown in between them, namely in such a way that the distances a to “home” (local time at the home location) and b to “clock” (local time at the destination) are, within the scope of the display resolution, proportional to the respective time shifts relative to these local times. In particular b corresponds to the actual time shift Δt of the pump time with respect to the local time at the destination or, in other words, to the time shift difference between the time shift between the home location and the destination and the current time shift of the diurnal profile of delivery which is currently being used for delivery of insulin with respect to the diurnal profile of delivery which was previously used at the home location.

FIG. 5a shows the situation shortly after the start of the changeover of the pump clock to the local time at the destination (the basal insulin delivery is still largely governed by the home time), FIG. 5b the situation in the middle during the changeover phase, and FIG. 5c the situation at the end of the changeover phase (basal insulin delivery is already largely governed by the local time at the destination). The pictogram “discharge profile” accordingly moves during the changeover phase from the pictogram “home” (local time at the home location) to the pictogram “clock” (local time at the destination).

For illustrative purposes, FIG. 6 shows the illustration analogous to FIG. 5a, for a journey in a westward direction (negative time shift).

In case the adaptation of the insulin pump to the time shift takes place in relatively fine time steps (for example hourly or at another selected interval), then at the start and towards the end of the changeover there is an overlap between the pictogram “discharge profile” and the pictograms “home” and “clock”. In this case, the overlapping pictograms can, for example, be displayed such that they flash alternately. Basically, the overlap can be avoided by vertically offsetting the movable pictogram “discharge profile” above or below the pictograms “home” and “clock”.

The local times at the home location and at the destination can optionally be displayed, for example, above or below the respective pictograms, thus making it possible to provide a clock with two time zones at the same time. Numerous further variants for visualization of the time shifts are likewise conceivable.

Since frequently with a specific interval between them two time shifts have to be carried out by the same amount but with opposite mathematical signs, for example at a holiday outward and return journey, the insulin pump furthermore has a specific “return journey command”, by which automatically the step-by-step time shift for the return journey is carried out without the time shift having to be reprogrammed. If further time shifts occur during the journey itself, for example, during long holiday journeys with intermediate stops, which furthermore are possible in both directions, then all of the time shifts which occur during the journey are added up with the correct mathematical signs and are together reversed by the “return journey command”, so that this automatically results in a changeover back to the home time.

Particularly when a plurality of time shifts follow one another, it often occurs that these superimpose each other, that is to say the respective next time shift takes place before the adaptation to the previous time shift has been completed. This superimposition is automatically taken into account in the following time shift. For example, if a changeover to a time shift of plus 4 hours (journey in an eastward direction) has already half been carried out (Δt=2 hours) and the rest of the journey results in a further time shift of plus 1 hour, then a changeover to a time shift of plus 3 hours must still be carried out.

An exemplary insulin pump according to the present invention offers the capability to automatically conduct the calculation of meal-time boli and correction boli. The conversion constants which are required for this are typically time-dependent, that is to say they follow, like the basal insulin demand, a time-of-day-dependent profile. They are, therefore, adapted in steps as when a time zone change takes place, in each case together with the shifting of the basal delivery profile. Since following to a relatively long journey, for a specific changeover phase a poor metabolism setting must be expected, which, however, can be tolerated for a limited time, corrections in both directions (in particular via additional insulin deliveries or by the consumption of glucose) should be carried out in this phase only with caution, since excessively frequent and strong corrections can lead to metabolism instability. Thus, in the course of the automatic calculation of the boli, the “target corridor” of the blood glucose which is aimed at by the calculation is for a limited time defined more broadly, i.e., higher or, if applicable, also lower blood glucose values, than otherwise without any correction. The return to the original values takes place automatically in dependence on specific stored parameters, such as the overall duration of the journey, either in one step, for example, at the end of the time changeover phase, or in steps.

In one embodiment, an insulin pump according to the present invention may comprise a special “travel menu” for programming travel-dependent time shifts, in which specific inputs are requested in dependence on the time shift that has been entered. In the event of a changeover by small amounts (for example, time shifts of up to 2 hours), as are carried out for occasional correction of a mistake or when a change to summer time takes place, the changeover is carried out without step-by-step adaptation. In the event of a changeover by a greater amount, an inquiry is automatically carried out to determine whether (and if appropriate with which parameters) a step-by-step adaptation of the basal insulin delivery should be carried out. Since a travel-dependent time changeover is, in practice, virtually the only reason for major time shifts in the continuous operation, provision is also made for the functionality discussed herein to be integrated directly into the time setting. In this case, the difference between the local time at the home location and the local time at the destination can be stored to allow the time shift to be cancelled by an “undo command”. This corresponds to the before mentioned “return journey command”. This command can also be used for a simple changeover of the pump clock between summer time and winter time.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A method for operation of a metering apparatus for liquid medicaments with a computer-aided control in the event of a travel-dependent shift in the local time, comprising the steps of:
a) controlling the metering apparatus with the control at a first location with a first local time (t1) such that it automatically delivers the liquid medicament in accordance with a first diurnal profile of delivery;
b) moving the metering apparatus to a second location with a second local time (t2), which is shifted by a first time shift with respect to the first local time (t1); and
c) controlling the metering apparatus with the control directly subsequent to the delivery in accordance with the first diurnal profile of delivery on a temporary basis such that it automatically delivers the liquid medicament in accordance with at least one further diurnal profile of delivery which differs from the first diurnal profile of delivery and from the first diurnal profile of delivery shifted by the first time shift, wherein the at least one further diurnal profile of delivery is determined by the control in dependence on the first time shift.

2. The method according to claim 1, wherein the metering apparatus is, after the second location, moved to a third location with a third local time, which is shifted by a second time shift relative to the second local time (t2), and the metering apparatus temporarily is controlled by the control such that it automatically delivers the liquid medicament in accordance with at least one further diurnal profile of delivery, which differs from the diurnal profile of delivery last used at the second location and from the first diurnal profile of delivery shifted by the sum, with the correct mathematical sign, of the first and the second time shift, wherein the at least one further diurnal profile of delivery is determined by the control in dependence on the second time shift.

3. The method according to claim 2, wherein the further diurnal profile of delivery, which has last been used at the second location deviates from a diurnal profile of delivery which corresponds to the first diurnal profile of delivery shifted by the first time shift, and wherein the deviation is taken into account in the determination of the further diurnal profile of delivery, which is determined in dependence on the second time shift.

4. The method according to claim 1, wherein part or all of further diurnal profiles of delivery correspond to a first diurnal profile of delivery which has been shifted in time by a specific time shift.

5. The method according to claim 4, wherein at least the further diurnal profiles of delivery which follow the diurnal profile of delivery which has last been used at the second location each correspond to a timewise shifted first diurnal profile of delivery, and the diurnal profile of delivery which has last been used at the second location corresponds to a first diurnal profile of delivery which has been shifted merely by a partial amount of the first time shift, and wherein the difference between this partial amount and the total amount of the first time shift is taken into account in the determination of the further diurnal profiles of delivery which are determined in dependence on the second time shift by being added with the correct mathematical sign to the second time shift.

6. The method according to claim 4, wherein the time shift difference between in each case two directly successive diurnal profiles of delivery, which correspond to the first diurnal profile of delivery or to a first diurnal profile of delivery which has been shifted in time by a specific time shift, corresponds to an amount of time which has been obtained by integer division of a time shift which has been used for determination of at least one of the diurnal profiles of delivery.

7. The method according to claim 6, wherein the amount of time is one of less than or equal to 4 hours or less than or equal to 3 hours.

8. The method according to claim 6, wherein the time shift differences between all of the directly successive diurnal profiles of delivery which correspond to the first diurnal profile of delivery or to a first diurnal profile of delivery which has been shifted in time by a specific time shift are identical.

9. The method according to claim 4, wherein a desired or maximum permissible time shift difference between directly successive diurnal profiles of delivery which correspond to the first diurnal profile of delivery or to a first diurnal profile of delivery which has been shifted in time by a specific time shift is communicated to the control by one of a manual input or via an interface and wherein the further diurnal profiles of delivery are determined by the control in dependence on the first or the second time shift and on the desired or maximum permissible time shift difference.

10. The method according to claim 9, wherein the interface is one of wireless or stored in the control.

11. The method according to claim 9, wherein part or all of the further diurnal profiles of delivery correspond to a first diurnal profile of delivery which has been compressed and/or stretched in time essentially continuously variably or at one or more points.

12. The method according to claim 11, wherein the stretching or compression is carried out in intervals by a specific amount of time.

13. The method according to claim 12, wherein the intervals are one of each hour, every 2 hours, every 4 hours or every 6 hours, and wherein the amount of time at all of the stretches or compressions is identical or, seen over the diurnal profile of delivery, stretches and/or compressions with different amounts of time are carried out.

14. The method according to claim 12, wherein all of the stretched or compressed diurnal profiles of delivery have an identical profile shape and are shifted in time with respect to one another.

15. The method according to claim 14, wherein a desired or maximum permissible amount of time for each stretch or compression and/or a desired or maximum and/or minimum permissible number of stretching or compression steps and/or a desired or maximum and/or minimum permissible interval between the stretching and compression steps is communicated to the control by a manual input or by an interface, which is in particular wireless, or is stored in the control and by means of the control further diurnal profiles of delivery are determined automatically in dependence on the first time shift and on the desired or permissible amount of time per stretch or compression and/or on the desired or maximum and/or minimum permissible number of stretching or compression steps and/or on the desired or maximum and/or minimum permissible interval between the stretching or compression steps.

16. The method according to claim 1, wherein at least part of the further diurnal profiles of delivery correspond to a first diurnal profile of delivery which has been shifted in time by a specific time shift and has at the same time been compressed or stretched in time.

17. The method according to claim 1, wherein a diurnal profile of delivery which follows the further diurnal profile or profiles of delivery corresponds to the first diurnal profile of delivery shifted by the first time shift or by the sum of the first and the second time shift.

18. The method according to claim 1, wherein the first and, if appropriate, the second time shift is communicated to the control by a manual input or by data transmission via an interface, which is wireless, or is determined by the control by evaluation of data received via a satellite navigation system.

19. The method according to claim 1, wherein a computer-aided control having at least two separate clocks, which are clocked jointly, is used such that, by a first of the clocks, the second local time or a third local time is administered and is displayed via a display at the metering apparatus, and, by a second of the clocks, the metering apparatus is controlled in accordance with a diurnal profile of delivery, wherein the second of the clocks is automatically set by the control such that it has a time offset with respect to the first local time which is less than the first time shift or the sum of the first and the second time shift.

20. The method according to claim 19, wherein the second of the clocks (4) is automatically set by the control such that it has a time offset with respect to the first local time which corresponds to the time shift of a further diurnal profile of delivery which is currently used to control the metering apparatus with respect to the first diurnal profile of delivery.

21. The method according to claim 20, wherein a degree of changeover of operation of the metering apparatus to the second local time or the third local time is displayed graphically on the metering apparatus, wherein at least one of the distance between graphical elements or the extent of graphical elements serves to illustrate time differences.

22. The method according to claim 20, wherein the relationship of the first local time, the second local time and/or the third local time as well as the actual degree of changeover of the operation of the metering apparatus to the second local time or to the third local time is illustrated graphically by pictograms, wherein distances to each other serve for the illustration of the respective time differences and are proportional to the respective time differences.

23. The method according to claim 22, wherein the actual degree of changeover of operation of the metering apparatus is illustrated in that the actual time of the second clock is displayed in relationship to the second local time, the third local time, and, additionally, the first local time.

24. The method according to claim 23, wherein the second local time and/or the third local time and, additionally, the first local time are displayed as one of an analogue or digital clock display at the metering apparatus.

25. The method according to claim 21, wherein by the metering apparatus additionally boli are calculated in dependence on the current degree of changeover of operation of the metering apparatus to the second local time or the third local time.

26. The method according to claim 25, wherein the boli are calculated such that a greater deviation from the otherwise normal target value or target value corridor of a parameter is permitted in the changeover phase.

27. The method according to claim 21, wherein control data for the metering apparatus are stored as a control history with reference to the first local time, wherein the second local time or the third local time is additionally stored at least on storage of the data relating to therapy-relevant information.

28. The method according to claim 1, wherein the metering apparatus is associated with a computer-controlled insulin pump.

29. A metering apparatus for liquid medicaments, comprising a feed device for moving the liquid medicament and a controller comprising a computer for automatically controlling the feed device in accordance with at least one profile of delivery.

30. The apparatus according to claim 29, wherein the at least one profile of delivery comprises a first diurnal profile of delivery stored in the controller, wherein the controller can determine at least one further diurnal profile of delivery, which differs from the first diurnal profile of delivery.

31. The apparatus according to claim 30, wherein the at least one further diurnal profile differs from the first diurnal profile by a first time shift.

32. The apparatus according to claim 31, wherein the at least one further diurnal profile is determined in dependence on the first time shift, and the feed device is controlled automatically in accordance with the at least one further diurnal profile of delivery.

33. A metering apparatus for liquid medicaments, comprising a feed device for feeding the liquid medicament and a computer-aided control for automatic control of the feed device in accordance with a first diurnal profile of delivery stored in the control, wherein in the event of a first time shift, at least one further diurnal profile of delivery which differs from the first diurnal profile of delivery and from the first diurnal profile of delivery which has been shifted by the first time shift can be determined in dependence on the first time shift and the feed device is automatically controlled by the control in accordance with the at least one further diurnal profile of delivery.

34. The metering apparatus according to claim 33, wherein in the event of a second time shift following the first time shift, at least one further diurnal profile of delivery, which differs from the last used diurnal profile of delivery and from the first diurnal profile of delivery shifted by the sum, with the correct mathematical sign, of the first and the second time shift, can be determined in dependence on the second time shift, and the feed device can be controlled automatically in accordance with the at least one further diurnal profile of delivery which has been determined in dependence on the second time shift.

35. The metering apparatus according to claim 34, wherein if the diurnal profile of delivery which has last been used for the purpose of adaptation to the first time shift deviates from the first diurnal profile of delivery which has been shifted by the first time shift, this deviation can be taken into account automatically in addition to the second time shift in the determination of the at least one further diurnal profile of delivery which is determined in dependence on the second time shift.

36. The metering apparatus according to claim 35, wherein further diurnal profiles of delivery can be determined and be used for automatic controlling, which correspond to a first diurnal profile of delivery which has been shifted in time by a specific time shift.

37. The metering apparatus according to claim 36, wherein a time shift difference which exists when determining the further diurnal profile of delivery in dependence on the second time shift between the first time shift and the time shift of the last used diurnal profile of delivery is automatically taken into account in the determination of the at least one further diurnal profile of delivery, said time shift difference is added with the correct mathematical sign to the second time shift.

38. The metering apparatus according to claim 37, wherein the time shift difference between two directly successive diurnal profiles of delivery, which correspond to the first diurnal profile of delivery or a first diurnal profile of delivery which has been shifted in time by a specific time shift, can be automatically adjusted to correspond to an amount of time which has been obtained by integer division of the time shift used for determination of one of the diurnal profiles of delivery, and wherein the control automatically limits this time shift difference to one of an amount of less than or equal to 4 hours or less than or equal to 3 hours.

39. The metering apparatus according to claim 38, wherein the time shift differences between all of the directly successive diurnal profiles of delivery which correspond to the first diurnal profile of delivery or to a first diurnal profile of delivery which has been shifted in time by a specific time shift can be set to be identical.

40. The metering apparatus according to claim 39, wherein a desired or maximum permissible time shift difference between directly successive diurnal profiles of delivery which correspond to the first diurnal profile of delivery or to a first diurnal profile of delivery which has been shifted in time by a specific time shift can be communicated to the control by one of a manual input or via an interface, which is in particular wireless, or is stored in the control, and the further diurnal profile of delivery can be determined by the control in dependence on the first or the second time shift and on the desired or permissible time shift difference.

41. The metering apparatus according to claim 40, wherein the interface is one of wireless or stored in the control.

42. The metering apparatus according to claim 40, wherein further diurnal profiles of delivery can be determined and be used for automatic controlling, which correspond to a first diurnal profile of delivery which has been compressed and/or stretched in time essentially in a continuously variable manner or at one or more locations.

43. The metering apparatus according to claim 42, wherein the stretching or compression is or can be carried out in intervals of one of selected intervals, each hour, every 2 hours, every 4 hours or every 6 hours, and the amount of time at all of the stretches or compressions can be or is set to be identical or, seen over the diurnal profile of delivery, different amounts of time for the stretches and compressions can be set or are set.

44. The metering apparatus according to claim 42, wherein several successive further diurnal profiles of delivery which have been stretched and/or compressed in time can be determined and be used for automatic controlling, which all have an identical profile shape and are shifted in time with respect to each other.

45. The metering apparatus according to claim 42, wherein at least one of a desired or maximum permissible amount of time for each stretching or compression process, a desired or maximum and/or minimum permissible number of stretching and/or compression steps, and a desired or maximum and/or minimum permissible interval between the stretching or compression steps can be communicated to the control by an interface or are stored in the control, and the further diurnal profiles of delivery can be determined by the control automatically in dependence on the first time shift and at least one of the desired or permissible amount of time per stretching or compression, on the desired or maximum and/or minimum permissible number of stretching or compression steps, and the desired or maximum and/or minimum permissible interval between the stretching or compression steps.

46. The metering apparatus according to claim 33, wherein further diurnal profiles of delivery can be determined and be used for automatic controlling, which correspond to a first diurnal profile of delivery which has been shifted in time by a specific time shift and has been compressed or stretched in time as well.

47. The metering apparatus according to claim 33, wherein a diurnal profile of delivery which follows the further diurnal profile or profiles of delivery determined by it corresponds to the first diurnal profile of delivery shifted by the first time shift or by the sum of the first and the second time shift.

48. The metering apparatus according to claim 33, wherein the respective time shift can be communicated to the control by a manual input or by data transmission via an interface or can be determined by the control by evaluation of data which are received via a satellite navigation system.

49. The metering apparatus according to claim 33, wherein the control comprises at least two separate jointly clocked clocks and administers with a first of the clocks a time which is shifted by the first or second time shift, and displays said time via a display at the metering apparatus, and controls the metering apparatus with the second of said clocks in accordance with a diurnal profile of delivery, wherein the second of the clocks, to control the metering apparatus in accordance with a further diurnal profile of delivery, can be adjusted automatically by the control whereby, before the first time shift, said second clock has a time offset with respect to a base time which is less than the first time shift or the sum of the first and the second time shift.

50. The metering apparatus according to claim 49, wherein the second of the clocks can be adjusted automatically via the control to have a time offset with respect to the base time which corresponds to the time shift of a further diurnal profile of delivery which is currently used to control the metering apparatus with respect to the first diurnal profile of delivery.

51. The metering apparatus according to claim 50, wherein the metering apparatus comprises a display by which a degree of adaptation of the operation of the metering apparatus to the first or second time shift can be displayed graphically, wherein one of the distance between graphical elements and the extent of graphical elements serve for the illustration of time differences.

52. The metering apparatus according to claim 51, wherein the relationship of the base time, of a time which has been shifted by the first time shift or the sum of the first and the second time shift with respect to the base time as well as the current degree of adaptation of the operation of the metering apparatus to the first or second time shift can be illustrated graphically by pictograms, whose distances to each other serve for the illustration of the respective time differences and are proportional to the respective time differences.

53. The metering apparatus according to claim 49, wherein the current degree of adaptation to the time shift can be illustrated by displaying and/or illustrating of the current time of the second clock in relationship to a time which has been shifted by the first time shift or the sum of the first and the second time shift with respect to the base time and can be displayed in addition to the base time.

54. The metering apparatus according to claim 33, wherein the time which has been shifted by the first time shift or the sum of the first and the second time shift with respect to the base time and the base time can be displayed at the metering apparatus as one of an analogue or digital clock display.

55. The metering apparatus according to claim 33, wherein the control is designed for additional calculation of boli, in dependence on the current degree of adaptation of the operation of the metering apparatus to the first or second time shift.

56. The metering apparatus according to claim 55, wherein the boli are calculated or can be calculated such that a greater deviation from the otherwise normal target value or target value corridor of a parameter is temporarily permitted in a changeover phase.

57. The metering apparatus according to claim 56, wherein the control records the control data of the metering apparatus as a control history with reference to the base time and a time shifted by the first time shift or the sum of the first and the second time shift with respect to the base time at least for the recording of the data of therapy-relevant information including bolus deliveries.

58. The metering apparatus according to claim 57, wherein the metering apparatus is used for the metered delivery of insulin.
